(19) United States
(12) Patent Application Publication
(10) Pub. No.: US 2021/0363562 A1
(43) Pub. Date: Nov. 25, 2021

(54) ONE-STEP FORMULATION FOR ALL-IN-ONE HYDROLYSIS SOLUTION

(71) Applicant: Kura Biotech, Inc., Rancho Dominguez, CA (US)

(72) Inventors: Manuel Rozas, Puerto Varas (CL); José Luis Callejas, Puerto Varas (CL); Camila Berner, Puerto Varas (CL); Mauricio Vargas, Puerto Montt (CL); Eduardo Wallach, Puerto Varas (CL); Jack Andrews, Youngsville, NC (US); Catalina Novoa, Puerto Varas (CL)

(73) Assignee: Kura Biotech, Inc., Rancho Dominguez, CA (US)

(21) Appl. No.: 17/324,067

(22) Filed: May 18, 2021

Related U.S. Application Data
(60) Provisional application No. 63/027,317, filed on May 19, 2020.

(51) Int. Cl. C12Q 1/34

(52) U.S. Cl. CPC C12Q 1/34; G01N 2333/938

(57) ABSTRACT

The present invention is related to a one-step formulation formulated for high-throughput analysis, long shelf-life, and stability. The kit includes an enzyme, β-glucuronidase enzyme derived from Brachyspira pilosicoli, in a formulation that meets the physicochemical requirements of reacting quickly and having stability at room temperature. The one-step formulation is intended for use in the detection of foreign substances within a subject's system.

### CROSS-REFERENCE TO RELATED APPLICATIONS

The present application claims the benefit of priority to U.S. provisional patent application No. 63/027,317, filed on May 19, 2020, entitled “One step mastermix enzyme kit and formulation for all-in-one hydrolysis solution.”

### TECHNICAL FIELD

The present invention is related with the technical field of biotechnology, and particularly discloses a one-step formulation for an all-in-one hydrolysis method for use in urine drug testing. The one-step formulation is formulated for high-throughput analysis, long shelf-life, and stability.

### BACKGROUND

Glucuronidation is a process in which glucuronic acid is bound to a substance, such as xenobiotics, drugs or any other metabolite such as some hormones, to allow for a more efficient transport, removal, or excretion of the substance from the organism when needed. Glucuronidation is one of the metabolic pathways that the human body uses to metabolize foreign substances or drugs. This process involves UDP-glucuronosyltransferase, an enzyme that transfers glucuronide from UDP-glucuronic acid onto the structure of the foreign substances. The glucuronide tag typically endows the substance with increased water solubility and thus allows for a more efficient excretion from the body. The glucuronide-tagged substance is found in the blood and urine of the body.

The glucuronide tag can complicate the accurate analysis of some-substances' content from a patient's sample (blood and/or urine). Therefore, β-glucuronidase is used to cleave the glucuronide tag from the substance so that the substance can be analyzed and quantified without the tag. The substance is detected and quantified via liquid chromatography-coupled to mass spectrometry (LCMS), high resolution mass spectrometry (HRMS), time of flight mass spectrometry (TOF-MS), ultraviolet-visible spectrophotometry (UV/Vis), flame ionization detection (FID), photodiode array detection (DAD), gas chromatography (GC), liquid chromatography (LC), high-performance liquid chromatography (HPLC), solid phase microextraction (SPME), or any other suitable separation method.

A typical protocol for the β-glucuronidase hydrolysis of a sample involves incubation of a sample containing a drug or any other glucuronide substance with β-glucuronidase at a prescribed temperature. The incubation temperature and time vary depending on the source of the β-glucuronidases, the specific drug or substances present in the sample, and the number of glucuronide tags that are appended to the drugs or substances.

Because the degree of modification of a drug may vary among individuals and drug type, urine drug testing labs typically aim for full cleavage of glucuronide-conjugated drugs before running a quantitation analysis by LC-MS/MS. New automated technologies and the increase in number of samples for processing in urine drug testing labs (e.g. to control use and misuse of prescription drugs) require new β-glucuronidases compatible with high throughput analysis.

Therefore, a need exists for a urine drug test utilizing β-glucuronidases capable of performing full hydrolysis of conjugates in a short period of time and ideally at room temperature.

The present invention relates to an all-in-one hydrolysis solution with glucuronidase activity and is useful in the field of toxicology, diagnostics, clinical settings, and medical tests for quick detection of drugs and toxic compounds by enzymatic reaction tests and kits, specifically for detection of opiates and opioids such as codeine glucuronide, from biological fluids, including urine.

The invention disclosed herein achieves this goal by providing a technology that includes an enzyme kit and a formulation that could meet the physicochemical requirements of this high-throughput enzyme: fast and stable at room temperature.

### SUMMARY

The present invention discloses a one-step master mix enzyme kit. The one-step master mix enzyme kit includes a β-glucuronidase enzyme, a buffer formulation, and purified water. The one-step master mix enzyme kit avoids the need for regular steps for enzyme kit detection, including mixing, cleaning, and heating steps. The one-step master mix enzyme kit is stable at room temperature or 20° C. for up to at least 3 months with no loss of hydrolysis activity and no detectable contamination, including bacterial, fungal, yeast or any other detectable microorganisms.

An embodiment of the present invention is a one-step formulation comprising a β-glucuronidase enzyme, tris (hydroxymethyl) aminomethane hydrochloride (Tris-HCl), ethylenediamine tetraacetic acid (EDTA), sodium phosphate, azide, and water. The β-glucuronidase enzyme has an amino acid sequence with at least 70% identity to SEQ. ID. No. 1.

An embodiment of the present invention is a one-step formulation comprising a β-glucuronidase enzyme, tris (hydroxymethyl) aminomethane hydrochloride (Tris-HCl), ethylenediamine tetraacetic acid (EDTA), sodium phosphate, azide, and water. The β-glucuronidase enzyme has an amino acid sequence with at least 70% identity to SEQ. ID. No. 2.

An embodiment of the present invention is a one-step formulation comprising a β-glucuronidase enzyme, tris (hydroxymethyl) aminomethane hydrochloride (Tris-HCl), ethylenediamine tetraacetic acid (EDTA), sodium phosphate, azide, and water. The β-glucuronidase enzyme has an amino acid sequence with at least 70% identity to SEQ. ID. No. 3.

Another aspect of the present invention includes a method of using a one-step formulation. The method comprises the steps of providing a sample, adding the one-step formulation to the sample, incubating the sample with the one-step formulation for a specific time period, and detecting a product using an analysis detection method. Analysis detection methods include LC-MS, GC-MS, HPLC-MS, LC-HRMS, HPLC-HRMS, LC-MS-MS, GC-MS-MS, and HPLC-MS-MS.

### BRIEF DESCRIPTION OF DRAWINGS

FIG. 1 graphically depicts features of the invention.

FIG. 2 graphically depicts example of the preferred hydrolysis protocol to be used with the invention.

FIG. 3 graphically depicts an example of storage time (days) at room temperature and relative activity (%) measure of the invention.

FIG. 4 graphically depicts an example of storage time (days) at room temperature and relative activity (%) measure of the invention.

FIG. 5 graphically depicts an example of storage time (days) at room temperature and relative activity (%) measure of the invention.

FIG. 6 graphically depicts an example of storage time (months) at varying temperatures and relative activity (%) measure of the invention.

FIG. 7 graphically depicts an example of storage time (months) at varying temperatures and relative activity (%) measure of the invention.

### DETAILED DESCRIPTION

The following description of the embodiments (e.g. including variations of embodiments, examples of embodiments, other suitable variants, etc.) is not intended to be limited to these embodiments, but rather to enable any person skilled in the art to make and use the disclosed technology.

“Purified water” as used herein may refer to any form of water with a reduced level of contaminants. This may refer to deionized water, sterilized water, distilled water, or filtered water.

“β-glucuronidase enzyme” as used herein may refer to any enzyme, including any other variants with functional activity, with the ability to catalyze the hydrolysis of a glycosidic bond.

“Glucuronide metabolite” as used herein may refer to any compound conjugated with glucuronic acid through a glycosidic bond. It is to be understood that a glucuronide metabolite can be natural or synthetic, and produced by chemical or enzymatic methods. The terms “conjugated compound,” “conjugated metabolite,” “conjugated drug,” conjugated metabolite,” “glucuronide compound,” and “glucuronide substrate” are used interchangeably.

“Products derived from glucuronide metabolites” or “recovered analytes” should be understood as the or original compound or molecule that is produced after hydrolysis or glycosidic bond cleavage of a glucuronide metabolite. These derived products or original compounds can be natural or synthetic, and produced by chemical or enzymatic methods.

“Enzyme” should be understood as a sequence of amino acids which form a functional protein of interest for the present invention. It is to be understood that the present invention is not related with enzymes in their natural environment, but it relates to proteins in an isolated state, purified or partially purified, or recombinant, obtained by any method of genetic engineering known in the state of the art. Likewise, it is to be understood that the term “variant,” “mutant” or “mutant enzyme” refers to a modified protein derived from the original amino acid sequence maintaining its β-glucuronidase enzymatic activity; and sharing at least 70%, preferably 80% and more preferably 90% of identity with the original sequence.

The term “identity” among amino acid sequences refers to the percent of identical amino acids that the compared sequences share among them, in a particular sequence alignment window. The percentage of identity can be calculated using a sequence comparison algorithm or by manual alignment together with visual inspection. For example, sequences and percentages of identity can be obtained using computer resources available on the internet such as BLAST (http://blast.ncbi.nlm.nih.gov/) or FastDB computer programs.

The term “sample” refers to any bodily fluid taken from a patient or specimen of interest, and any mixture, of synthetic or natural origin, that may contain glucuronide substance. The bodily fluid is chosen from the group consisting of saliva, whole blood, plasma, urine, hair, skin, teeth, soft tissue, water, food, meconium, vitreous humour, bile, earwax, phlegm, vomit, aqueous humour, tears, amniotic fluid, vaginal secretion, semen, pre-ejaculate fluid, mucus, sebum, sweat, excrement, or any other suitable sample without limitation to the animal fluids previously mentioned.

It should be understood by one of ordinary skill in the art that the method of this invention can be implemented via various quantitative laboratory instruments, including but not limited to mass spectrometry (MS), tandem mass spectrometry (MS/MS), high resolution mass spectrometry (HRMS), time of flight mass spectrometry (TOF-MS), ultraviolet-visible spectrophotometry (UV/Vis), flame ionization detection (FID), photodiode array detection (DAD), gas chromatography (GC), liquid chromatography (LC), high-performance liquid chromatography (HPLC), solid phase microextraction (SPME), or any other suitable separation method.

The terms “Unit”, “U” and “Activity Unit” are used interchangeably and refer to an experimentally determined parameter defining the amount of glucuronidase enzyme that is required to cleave the glycosidic bond in a certain amount of standard glucuronide substance or model substrate in a given amount of time under controlled pH and temperature conditions. The terms “PS-U” and “Product-Specific Units” refer to activity units measured with a model substrate under the same pH, temperature and time conditions specified and recommended for a particular glucuronidase enzyme; this PS-U herein described, is part of the unity of measure of the present invention, regarding an internal standard for the measure of the activity of the enzyme used and developed within the invention.

Here we describe a one-step formulation and one-step method of use thereof in the detection of products derived from glucuronide metabolites present in a sample including a blood and/or urine sample.

This one-step formulation reduces the number of steps by negating the need for mixing, cleaning, and heating steps. The one-step formulation is stable at room temperature (20° C.) for up to at least 3 months with no loss of hydrolysis activity and no detectable contamination, including bacterial, fungal, yeast or any other detectable microorganisms.

The one-step formulation can be used in modern high-throughput laboratories that use automated liquid dispenser instruments. The one-step formulation provides increased hydrolytic activity for a broad spectrum of conjugated analytes, including but not limited to “hard to cleave” glucuronides, specifically opiates and opioids such as codeine glucuronide. Its capacity for immediate hydrolysis, without a heating step, enables modern toxicology laboratories to eliminate sample preparation bottlenecks while enabling fully automated hydrolysis and same-day reporting of accurate and reproducible analytical results.

The one-step formulation can be stored at 4° C. for at least 18 months. Additionally, the one-step formulation does not need to be refrigerated before, during, or after use due to its stability at room temperature.

The enzyme of the one-step formulation is a recombinant enzyme providing certainty in the metabolization process, including metabolizing β-glucuronides.

The one-step formulation and method of using one-step formulation can eliminate routine steps/bottlenecks, including preparing a buffer solution, measuring pH, having heating devices, centrifuges, using time for incubation, crashing steps, and any combination of them.

The one-step formulation and one-step method can reach high recoveries for opiates (e.g. codeine), in a short period of time at room temperature, wherein room temperature can comprise any temperature in the range of greater than 18° C. and less than 26° C.

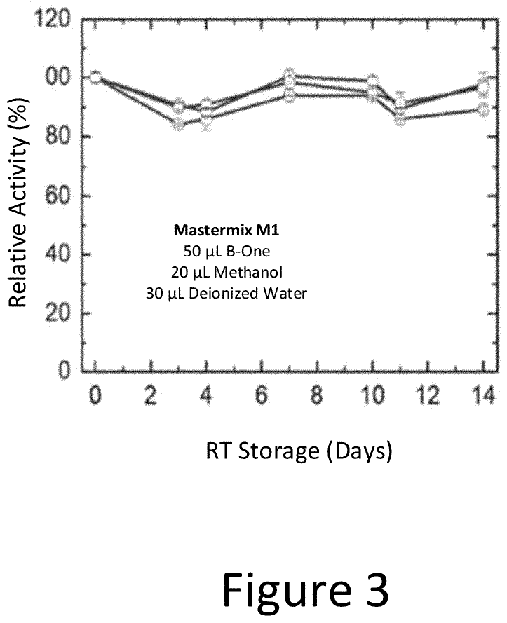

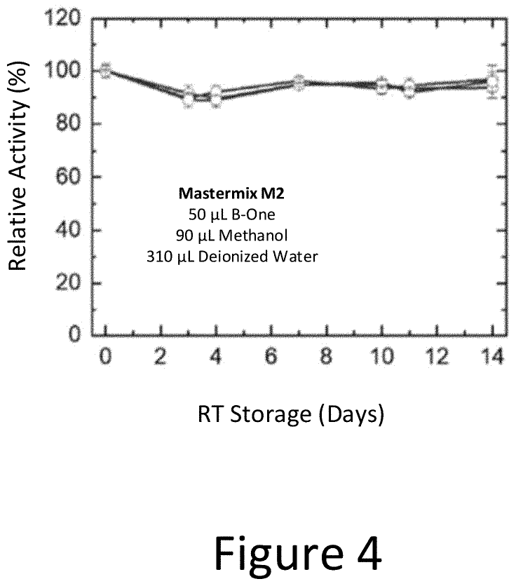

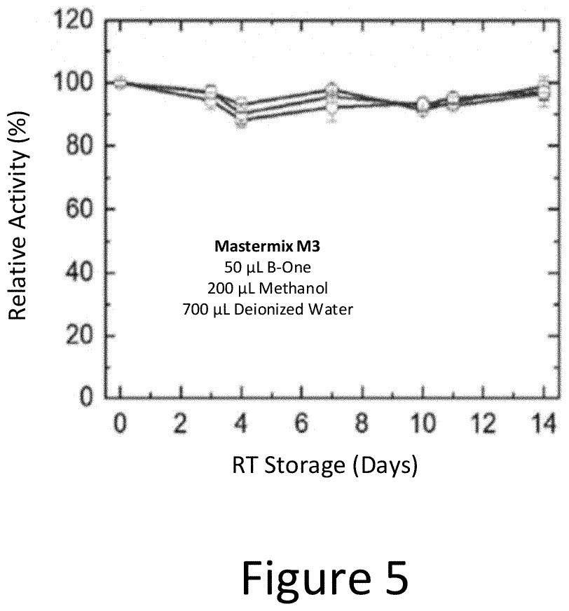

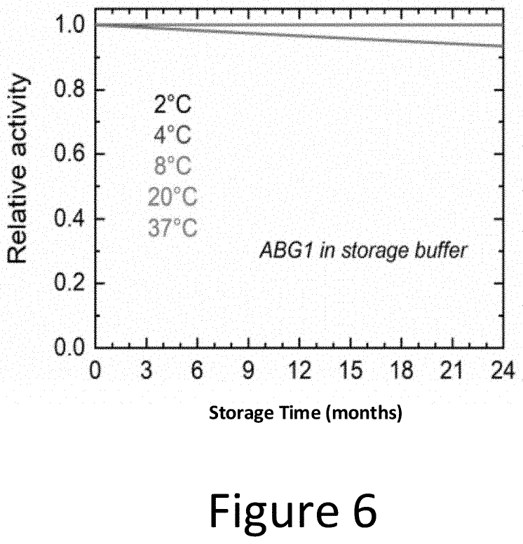

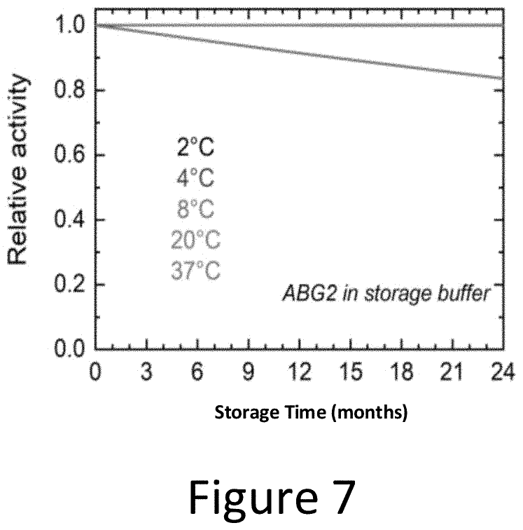

The one-step formulation can tolerate medium and long-term situations related with logistic and transport, wherein the toleration parameters can include as follows: temperature can be tolerated in the range of more than 40° C. and less than 26° C. Transport conditions includes up to 10 days in standard conditions without a temperature-controlled environment, and over 10 days up and to 1 year in a temperature-controlled environment with refrigeration, and over 10 days and up to 3 months in a temperature-controlled environment without refrigeration, without compromising the quality of the product. Additionally, or alternatively, the embodiment of the invention can allow long-term transport times across the world, in any shipping method available and transport, under a specific range of temperature during the transport or storage process, for a certain period of time. This embodiment of the invention allows to provide an enzymatic kit in emergency situations that increase the time of transport and logistics, reducing cost and loss of quality of the one-step formulation. As shown in FIG. 3, the β-glucuronide of SEQ. ID. No. 1, retains activity for more than 14 days in a mastermix (M1). As shown in FIG. 4, the β-glucuronides of SEQ. ID. No. 1, retains activity for more than 14 days in a mastermix (M2). As shown in FIG. 5, the β-glucuronides of SEQ. ID. No. 1, retains activity for more than 14 days in a mastermix (M3). As shown in FIG. 6, the β-glucuronide of SEQ. ID. No. 2, retains activity for more than 24 months in a storage buffer. As shown in FIG. 7, the β-glucuronide of SEQ. ID. No. 2, retains activity for more than 24 months in a storage buffer.

The one-step formulation can facilitate a streamline workflow, allowing a completely hands-free processing of samples and provides the potential to completely automate the sample preparation process.

The one-step formulation comprises an “One-step Hydrolysis” process, that provides added robustness to the assay, and saves prep work increasing the efficiency of urine drug testing or UDT.

The one-step formulation, in combination with a liquid handler and Tip-On-Tip Technology creates the synergy for complete automation of UDT. The one-step formulation in combination with Tip on Tip Technology provides an easier workflow and cleaner samples for improved assay robustness.

The one-step formulation allows at least 85% of recovery of codeine within at least 5 minutes (or less) to 10 minutes, providing a quick UDT for the detection of codeine.

The one-step formulation can be used as part of or directly as a point of care (POC) kit, for codeine or any other glucuronide-conjugated compound, able to react and be hydrolyzed by the one-step formulation.

The one-step formulation can include an additional enzyme to allow for the detection of any substrate-conjugated compound, wherein the substrate of the substrate-conjugated compound is a target of the enzyme included in the kit.

The enzyme of the one-step formulation has a higher and increased activity, providing and efficient hydrolysis with a reduced concentration of enzyme, providing an improved process that avoids the obstruction of columns, eliminating the need to centrifuge or to do “Protein Crash/protein breakdown” or an extraction method to get rid of the protein/enzyme.

The one-step formulation incorporates a new enzyme with β-glucuronidase activity originated from genus Brachyspira, which has an improved affinity for glucuronide metabolites, a specific activity 6.6 times higher than β-glucuronidase originated from E. coli, and therefore it is capable of hydrolyzing glucuronide metabolites in periods of time as short as one minute, preferably between three and 30 minutes. This enzyme is preferably originated from species Brachyspira pilosicoli, whose amino acid sequence is shown in SEQ ID No. 1, but it can be obtained from any species of genus Brachyspira. Additionally, in the method of the present invention, any derivative or variant of this protein can be used, provided that it maintains its β-glucuronidase activity. Preferably, the derivative or variant enzyme with β-glucuronidase activity shares at least a 70% of sequence identity, more preferably an 80% of identity, and even more preferably a 90% of identity with the sequence defined in SEQ ID No. 1. Alternatively, the derivative or variant enzyme with β-glucuronidase activity shares at least a 70% of sequence identity, more preferably an 80% of identity, and even more preferably a 90% of identity with the sequence defined in SEQ ID No. 2. Alternatively, the derivative or variant enzyme with β-glucuronidase activity shares at least a 70% of sequence identity, more preferably an 80% of identity, and even more preferably a 90% of identity with the sequence defined in SEQ ID No. 3.

The β-glucuronidase enzyme originated from Brachyspira can hydrolyze any glucuronide metabolite, whether from natural, semi-synthetic or synthetic origin. Some examples of these metabolites, without limitation, are: morphine-3-glucuronide, morphine-6-glucuronide, codeine-6-glucuronide, oximorphone-glucuronide, hydromorphone-glucuronide, norbuprenorphine-glucuronide, buprenorphine-glucuronide, oxazepam-glucuronide, temazepam-glucuronide, lorazepam-glucuronide, alprazolam-glucuronide, midazolam-glucuronide, nordiazepam-glucuronide, psilocin-glucuronide, carboxy-tetrahydrocannabinol-glucuronide, tetrahydrocannabinol-glucuronide, tetrahydrocannabinolic acid-glucuronide, naloxone-3-glucuronide, tapentadol-glucuronide, tricyclic antidepressant-glucuronide, cannabidiol-glucuronide, among others. It is to be understood for the scope of the present invention that the β-glucuronidase enzyme originated from Brachyspira can hydrolyze any glucuronide metabolite and it is not limited to the examples previously mentioned.

An embodiment of the claimed invention comprises a one-step formulation comprising an enzyme, a mastermix solution and water. In some embodiments, the enzyme is a shelf-stable enzyme. In some embodiments, the buffer formulation comprises organic solvents, inorganic compounds, polar solvents, Tris-HCL, EDTA, sodium phosphate, and/or preservation additives such as sodium azide. In some embodiments, the water is sterilized water, distilled water, pure water, and or any other form of water reduced and/or free of contaminants.

In some embodiments, the one-step formulation comprises an enhanced recombinant β-glucuronidase that is liquid chromatography compliant and optimized to perform instant hydrolysis at room temperature with no need to add a buffer.

In a preferred embodiment, the one-step formulation comprises: an enzyme, a mastermix solution, and water, wherein the enzyme is an enhanced recombinant enzyme with β-glucuronide activity, wherein the mastermix solution can comprise: Tris-HCl in the range of 2-200 mM, EDTA in the range of 0.1-1 mM, sodium phosphate in the range of 2-200 mM, sodium acetate in the range of 0.001-200 mM, and sodium azide in the range of 0.001-0.1% w/v, with a final pH in the physiological range (4 to 10 pH), and wherein the water comprises: sterilized water, distilled water, pure water or any other form of water reduced and/or free of different compounds.

In another embodiment of the invention, the mastermix solution can comprise tris (hydroxymethyl) aminomethane hydrochloride (Tris-HCl) in a concentration between 2-200 mM, ethylenediamine tetraacetic acid (EDTA) in a concentration amount between 0.001 and 1 mM, and sodium azide in the range of 0.001-0.1% w/v.

In another embodiment of the invention, the master mix solution comprises: glycerol 50% w/v, sodium phosphate in a concentration amount between 2 and 200 mM and sodium azide in the range of 0.001-0.1% w/v.

In a preferred embodiment of the invention, the one-step formulation of can include the enzyme SEQ ID 1 and the master mix solution comprising: tris (hydroxymethyl) aminomethane hydrochloride (Tris-HCl) in a concentration between 2-200 mM, ethylenediamine tetraacetic acid (EDTA) in a concentration amount between 0.001 and 1 mM, sodium azide in the range of 0.001-0.1% w/v, and wherein the one-step master mix formulation has a final pH of 4 to 10.

In a preferred embodiment of the invention, the one-step formulation of can include the enzyme SEQ ID 2 and the master mix solution comprising: tris (hydroxymethyl) aminomethane hydrochloride (Tris-HCl) in a concentration between 2-200 mM, ethylenediamine tetraacetic acid (EDTA) in a concentration amount between 0.001 and 1 mM, sodium azide in the range of 0.001-0.1% w/v, and wherein the one-step master mix formulation has a final pH of 4 to 10.

In another embodiment of the invention, the master mix formulation can comprise a β-glucuronidase enzyme, tris (hydroxymethyl) aminomethane hydrochloride (Tris-HCl), ethylenediamine tetraacetic acid (EDTA), sodium phosphate, sodium azide, and water; and wherein the β-glucuronidase enzyme has an amino acid sequence with at least 70% identity to SEQ. ID. No. 1.

In another embodiment of the invention, the master mix formulation can comprise a β-glucuronidase enzyme, tris (hydroxymethyl) aminomethane hydrochloride (Tris-HCl), ethylenediamine tetraacetic acid (EDTA), sodium phosphate, sodium azide, and water; and wherein the β-glucuronidase enzyme has an amino acid sequence with at least 70% identity to SEQ. ID. No. 2.

In another embodiment of the invention, the master mix formulation can comprise a β-glucuronidase enzyme, tris (hydroxymethyl) aminomethane hydrochloride (Tris-HCl), ethylenediamine tetraacetic acid (EDTA), sodium phosphate, sodium azide, and water; and wherein the β-glucuronidase enzyme has an amino acid sequence with at least 70% identity to SEQ. ID. No. 3.

Preferably, the reagent contains an enzyme with β-glucuronidase activity originated from species Brachyspira pilosicoli, or any derived enzyme or mutant thereof. Said enzyme has an amino acid sequence shown in SEQ ID No. 1, but it can be any enzyme variant from said sequence and maintaining β-glucuronidase activity. Preferably, the derived or mutant enzyme with β-glucuronidase activity shares at least a 70% or 80% of identity with the sequence defined in SEQ ID No. 1, more preferably a 90% of identity with the sequence defined in SEQ ID No. 1.

Preferably, the reagent contains an enzyme with β-glucuronidase activity originated from species Brachyspira pilosicoli, or any derived enzyme or mutant thereof. Said enzyme has an amino acid sequence shown in SEQ ID No. 2, but it can be any enzyme variant from said sequence and maintaining β-glucuronidase activity. Preferably, the derived or mutant enzyme with β-glucuronidase activity shares at least a 70% or 80% of identity with the sequence defined in SEQ ID No. 2, more preferably a 90% of identity with the sequence defined in SEQ ID No. 2.

Preferably, the reagent contains an enzyme with β-glucuronidase activity originated from species Brachyspira pilosicoli, or any derived enzyme or mutant thereof. Said enzyme has an amino acid sequence shown in SEQ ID No. 3, but it can be any enzyme variant from said sequence and maintaining β-glucuronidase activity. Preferably, the derived or mutant enzyme with β-glucuronidase activity shares at least a 70% or 80% of identity with the sequence defined in SEQ ID No. 3, more preferably a 90% of identity with the sequence defined in SEQ ID No. 3.

In a preferred embodiment, the reagent contains between 0.01 and 10 mg/mL of enzyme. In addition to the β-glucuronidase enzyme, the reagent includes an appropriate vehicle that varies depending on the product format and on the function that the same performs. For example, the reagent including the enzyme can be in a liquid format, which could require only water as a vehicle.

To improve the stability of the enzyme, salts can be added (at μM or mM concentrations), or ethylenediamine tetraacetic acid (EDTA), Tris, dithiothreitol (DTT), hydroxyethyl piperazin-yl ethanesulfonic (HEPES), citric acid, among others, or a combination thereof. Preferably, salts are selected from the group consisting of sodium phosphate, potassium phosphate, sodium carbonate, sodium acetate, sodium citrate, sodium chloride and potassium chloride, without limitation to the salts previously described. In case that the reagent is frozen, the vehicle to be used can include glycerol, preferably at a concentration of 50%. On the other hand, if the reagent format is powder, which can be produced by means of lyophilization, spray drying or any other pulverization method; vehicles able to protect the enzyme from drying process are required such as sugars, amino acids and some salts, which are present at a concentration range between 0.001 to 2% based on liquid previous to the drying process. In this case, sugars are preferably selected from the group consisting of sorbitol, trehalose, sucrose, glucose, lactose, mannitol and raffinose, without limitation to these described examples. Therefore, the vehicle of the present invention fulfils the purpose of being a diluting, preserving, or stabilizing medium for the enzyme.

In another embodiment, internal standards can be added to the one-step formulation before using to prepare an internal standard mastermix providing enzymatic stability for up to 14 days at room temperature, eliminating stability issues and waste.

The enzymes used in the invention boast an activity rate (PS-U) between 5.000-800.000 U/mL, with 20,000 PS-U/mL, 100,000 U/mL, and 300,000 U/mL being most common.

In further embodiments, the invention comprises a method of using the one-step formulation wherein the one-step formulation is added to a sample. The method comprises the steps of:
a. providing a sample in which a product is to be detected,
b. adding the one-step formulation to the sample
c. incubating the sample with the one-step formulation for a determined length of time, and
d. detecting a product using a detection method.

The method allows for the reduction of such steps as mixing, cleaning, and heating that are typically required for other such protocols.

In a preferred embodiment, the volume range of enzyme to sample is from 1:1 to 1:100. In a more preferred embodiment, the optimum concentration of enzyme in the sample solution is between 0.02 to 0.25 mg of enzyme per mL of total reaction solution.

In an incubation step, the mixture comprising the sample and the one-step formulation are incubated for a prescribed amount of time in suitable conditions. The prescribed amount of time can be as little as one minute or as much as 16 hours or more. Suitable conditions include a temperature range between 20° C.-60° C. and a pH range between 4.0-9.0. A preferred temperature range is 50° C.-55° C. A preferred pH range is 4-10. In some embodiments, the incubation step is between 2 and 120 minutes. A preferred embodiment comprises an incubation step that is between 3 and 30 minutes.

In some embodiments, the incubation step is followed by an optional extraction step. The optional extraction step can be performed via liquid-liquid extraction, solid-liquid extraction, extraction in solid phase, or any other suitable extraction technique. In a further embodiment, the optional extraction step is followed by dilution with an appropriate solvent and loading the diluted sample into a separation column. The separation column can be configured for use with HPLC, LC, GC, or any other chromatographic technique.

In another embodiment, the incubation step is followed by dilution with an appropriate solvent and loading the diluted sample into a separation column. The separation column can be configured for use with HPLC, LC, GC, or any other chromatographic technique.

In some embodiments, the detection of products derived from glucuronide metabolites is performed through the use of separation methods and analytical techniques. In some embodiments these analytical techniques are selected from the group consisting of: mass spectrometry (MS), tandem mass spectrometry (MS/MS), high resolution mass spectrometry (HRMS), time of flight mass spectrometry (TOF-MS), ultraviolet-visible spectrophotometry (UV/Vis), flame ionization detection (FID), photodiode array detection (DAD), or any other suitable detection method. In some embodiments the separation methods are selected from the group consisting of gas chromatography (GC), liquid chromatography (LC), high-performance liquid chromatography (HPLC), solid phase microextraction (SPME), or any other suitable separation method. Embodiments include the use of LCMS, GCMS, HPLC-MS, SPME-MS, GC-HRMS, LC-HRMS, HPLC-HRMS, SPME-GC-MS/MS, LC-MS/MS, GC-MS/MS, LC-TOF-MS, GC-TOF-MS, HPLC-TOF-MS, GC-UV/Vis, LC-UV/Vis, SPME-UV/Vis, GC-FID, LC-FID, SPME-FID, LC-DAD, and any other suitable method.

In some embodiments, the method of detection of products derived from glucuronide metabolites is performed in a high-throughput manner. In some embodiments, the method of detection of products derived from glucuronide metabolites is performed in an automated manner. In some embodiments, the method of detection of products from glucuronide metabolites is performed in combination with “tip-on-tip” technology.

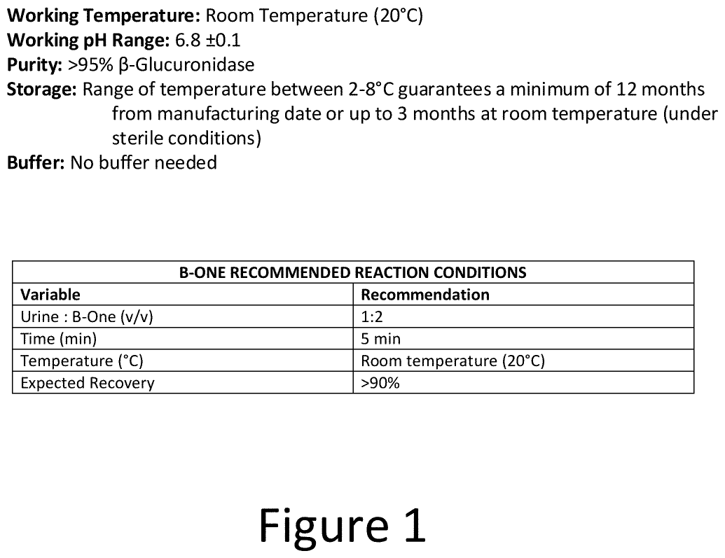

As an example of the invention, the recommended reaction conditions are shown in FIG. 1. Recommended reaction conditions include a working temperature of room temperature or 20° C. and a working pH of 6.8±1. Further recommended reaction conditions are a 1:2 ratio of sample (such as urine) to one-step formulation, and an incubation time of 5 minutes Following these recommended reaction conditions, the expected recovery is greater than 90%. Recommended storage conditions are also shown in FIG. 1. Under sterile conditions, the one-step formulation is stable up to 12 months at a temperature of 2-8° C., and stable up to 3 months at room temperature.

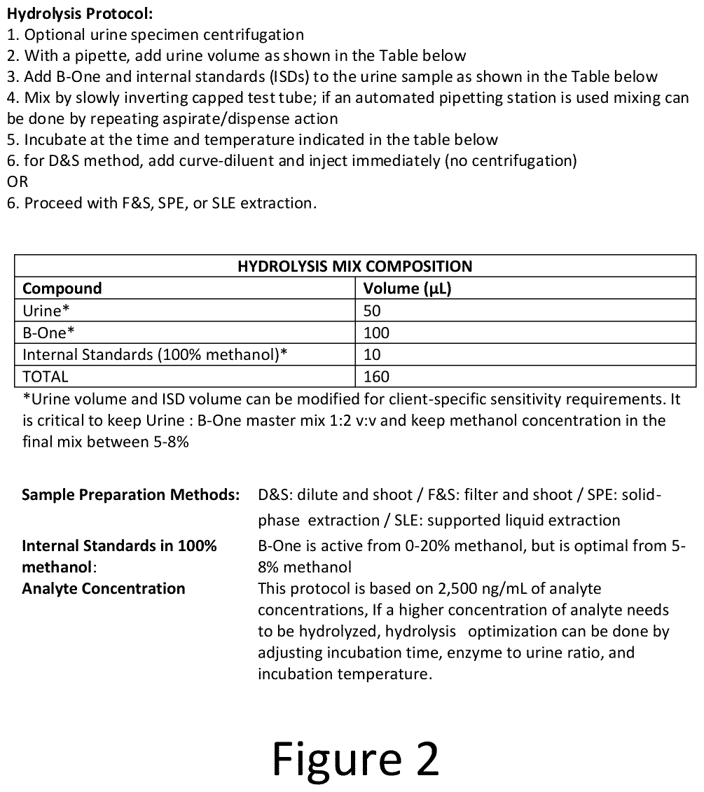

As an example of the invention, the invention comprises a hydrolysis protocol, shown in FIG. 2, recommended for enhanced the activity of the enzyme, or at least, ensure the effectiveness of its use and application. The hydrolysis protocol includes the optional step of centrifugation of the sample and mixing the sample with the one-step formulation and internal standards (ISDs). This is followed by an incubation period at the temperature indicated. For “dilute and shoot” or D&S methods, the curve-diluent can be added and injected into the appropriate analytical instrument immediately. For other methods, the mixture should be filtered and subjected to extraction, such as solid-phase extraction (SPE) or supported liquid extraction (SLE).

As an example of the invention, current technology can be mixed with methanol (MeOH) and stored at room temperature for at least 12 days without loss of activity, with at least or up to 90% of the activity of the enzyme within the kit (FIG. 1). As shown in FIG. 3, FIG. 4, and FIG. 5, the mixtures retain activity after two weeks of storage at room temperature.

The invention comprises one of the following sequences or variants thereof:

| SEQ ID NO. 1: This application herein incorporates the SEQ ID NO. 1 of US | |
|---|---|
| 2020/0116719 A1 directed to Brachyspira pilosicoli. | |
| SEQ ID NO. 2: Recombinant BRG-WT protected | |
| 1 | mvnsmlypre srtrrvvdis gmwefkidin negrnsgyan glkdttfipvpssfndlftd |
| 61 | knirehagdv wyetsfylpl ewkdkdvnvr fgcatheatv yingkevcthvggfmpfnap |
| 121 | vneagifgek nklvvvvnne lsnttipcgh tetkpsgkky ikpsfdffnyaglnrpvkit |
| 181 | vtnkeyihdi dilsdvngsd givnyevhtt genkvyikin deegkevascegksgkivik |
| 241 | daklwnpkaa ylykfiacik ngdelideyy ldfgirtvkv egtkflingkpfyftgfgkh |
| 301 | edseiagrgy nppvikrdfe likwvgansf rtshypysee imqaadregiviidevaavg |
| 361 | mfdygsvlnp sasktdyfsl devhsktkev hkkaveelik rdknhpsvvmwslfnepdts |
| 421 | kdeavpyfed ifnfaksqdk qnlpktfaai qasspgkckc mhlcdvitlnryygwyflgg |
| 481 | yeidmseekf reemnlysnm nkpvmfteyg adtyagyhkl psvmwseeyqceyyemnfkv |
| 541 | fdsydfivge qlwnfadfqt tegifrvdgn kkgiftrnrq pkavahlirsrwnklpldyk |
| 601 | skkklaaale hhhhhh |
| SEQ ID NO. 3: Recombinant ABG (BGT G563S) | |
| 1 | mvnsmlypre srtrrvvdis gmwefkidin negrnsgyan glkdttfipv pssfndlftd |
| 61 | knirehagdv wyetsfylpl ewkdkdvnvr fgcatheatv yingkevcth vggfmpfnap |
| 121 | vneagifgek nklvvvvnne lsnttipcgh tetkpsgkky ikpsfdffny aglnrpvkit |
| 181 | vtnkeyihdi dilsdvngsd givnyevhtt genkvyikin deegkevasc egksgkivik |
| 241 | daklwnpkaa ylykfiacik ngdelideyy ldfgirtvkv egtkflingk pfyftgfgkh |
| 301 | edseiagrgy nppvikrdfe likwvgansf rtshypysee imqaadregi viidevaavg |
| 361 | mfdvgsvlnp sasktdyfsl devhsktkev hkkaveelik rdknhpsvvm wslfnepdts |
| 421 | kdeavpyfed ifnfaksqdk qnlpktfaai qasspgkckc mhlcdvitln ryygwyflgg |
| 481 | yeidmseekf reemnlysnm nkpvmfteyg adtyagvhkl psvmwseeyq ceyyemnfkv |
| 541 | fdsydfivge qlwnfadfqt tesifrvdgn kkgiftrnrq pkavahlirs rwnklpldyk |
| 601 | skkklaaale hhhhhh |

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments described herein can be embodied and/or implemented at least in part as a machine configured to receive a computer-readable medium storing computer-readable instruction. The instructions can be executed by computer-executable components that can be integrated with the system.

As a person skilled in the art will recognize from the above detailed description and from the figures and claims, modifications and changes can be made to embodiments of the system herein described, and/or variants without departing from the scope defined in the claims.

1. A one-step formulation including an all-in-one hydrolysis solution for xenobiotic detection, comprising:
a enzyme;
a mastermix solution;
purified water; and
wherein said one-step formulation is stable at room temperature or 20° C. for up to at least 3 months with no loss of hydrolysis activity and no detectable contamination, including bacterial, fungal, yeast or any other detectable microorganisms.

2. The one-step formulation of claim 1, wherein said enzyme has an amino acid sequence with at least 70% identity to SEQ. ID. No. 1.

3. The one-step formulation of claim 1, wherein said enzyme has an amino acid sequence with at least 70% identity to SEQ. ID. No. 2.

4. The one-step formulation of claim 1, wherein said enzyme has an amino acid sequence with at least 70% identity to SEQ. ID. No. 3.

5. The one-step formulation of claim 1, wherein said enzyme is present in a concentration between 0.01-10 mg/mL.

6. The one-step formulation of claim 5, wherein said enzyme comprises an activity rate (PS-U) between 5.000-800.000 U/mL.

7. The one-step formulation of claim 1, wherein said mastermix solution of said one-step formulation comprises one or more components from the group consisting of ethylenediamine tetraacetic acid (EDTA), tris (hydroxymethyl) aminomethane hydrochloride (Tris-HCl), dithiothreitol (DTT), hydroxyethylpiperazin-ylethanesulfonic acid (HEPES), methanol, ethanol, potassium acetate, sodium formate, potassium formate, ammonium formate, sodium chloride, magnesium chloride, glycerol, imidazol, glycin, betaine, osmolytes, any other stabilization molecules including BSA, citric acid, sodium phosphate, potassium phosphate, sodium carbonate, sodium acetate, sodium citrate, sodium chloride, azide, any other suitable biocide agents, potassium chloride, any other chelating agents.

8. The one-step formulation of claim 1, wherein said master mix solution comprises: tris (hydroxymethyl) aminomethane hydrochloride (Tris-HCl) in a concentration between 2-200 mM, ethylenediamine tetraacetic acid (EDTA) in a concentration between 0.001 and 1 mM, sodium phosphate in a concentration between 2 and 200 mM, and azide in the range of 0.001-0.1% w/v, and
wherein the one-step formulation has a final pH of 4 to 10.

9. The one-step formulation of claim 1, wherein said master mix solution comprises: glycerol 50% w/v, sodium phosphate in a concentration amount between 2 and 200 mM and azide in the range of 0.001-0.1% w/v.

10. The one-step formulation of claim 1, comprising an enzyme, tris (hydroxymethyl) aminomethane hydrochloride (Tris-HCl), ethylenediamine tetraacetic acid (EDTA), sodium phosphate, azide, and water;
wherein the enzyme of claim 1 has an amino acid sequence with at least 70% identity to SEQ. ID. No. 3.

11. A method of using the one-step formulation of claim 1, wherein the method comprises the steps of:
providing a sample in which a product is to be detected,
adding the one-step formulation of claim 1 to the sample,
incubating the sample with the one-step formulation of claim 1 for a determined length of time, and
detecting a product using an analysis detection method, wherein the analysis detection method is selected from the group consisting of LC-MS, GC-MS, HPLC-MS, LC-HRMS, HPLC-HRMS, LC-MS-MS, GC-MS-MS, or HPLC-MS-MS, and
wherein the method utilizes an all-in-one hydrolysis solution to avoid the need to distinctly perform the separate steps for formulation detection, including the steps of mixing, cleaning, and heating the sample.

12. The method of the claim 11, wherein the method includes centrifuging the sample prior to the step of incubating the sample.

13. The method of the claim 11, wherein the method includes extracting the sample after incubating the sample.

14. The method of claim 11, wherein the one-step formulation is used to detect xenobiotic compounds, including drug detection and any other suitable metabolites derived from a glucuronide metabolism process, in a sample, wherein the sample is selected from the group consisting of urine, blood, and any other suitable organic sample for detection of toxic compounds by enzymatic reaction assay.
